SEQUENCE LISTING (8 sequences)

SEQ ID NO: 1 - protein, 363 aa, Cimex lectularius
MIIFALAALQLAAGCVYQGTDAGYANDATYFNGVANGLNTVSNDFNGVATGFNGVATGFNGVATGFNGVATGFNGVATGFNGVATGFDGVATDLNGVSTGFVNAAPATDAVDYVAGNNLDGVASGFVDNAAGYYNAPTTCLTHRLEQVVRPVVDECVRHVPVVRKTVRHQEEQFTRHVPVVEKTVSQQEEKFVRHVPVVQKYTRPVVETRTHHVPVVKHRTKTVVEPYTDHVPVTEKYTRPVEETVVRTVPVVERRTKPVVHQHVRHVPVTERRSRPVVESHVTTVPVKQRFVKNLVEQRVRSSSNPCAPCPPGFYSGVEGYAPASYATSSFAAPALGDVYTGSFAGHATETLPATYNTHATC

SEQ ID NO: 2 - protein, 14 aa, Cimex lectularius
RHVPVTERRSRPVV

SEQ ID NO: 3 - protein, 14 aa, Cimex lectularius
KYTRPVVETRTHHV

SEQ ID NO: 4 - protein, 250 aa, Cimex lectularius
VAGNNLDGVASGFVDNAAGYYNAPTTCLTHRLEQVVRPVVDECVRHVPVVRKTVRHQEEQFTRHVPVVEKTVSQQEEKFVRHVPVVQKYTRPVVETRTHHVPVVKHRTKTVVEPYTDHVPVTEKYTRPVEETVVRTVPVVERRTKPVVHQHVRHVPVTERRSRPVVESHVTTVPVKQRFVKNLVEQRVRSSSNPCAPCPPGFYSGVEGYAPASYATSSFAAPALGDVYTGSFAGHATETLPATYNTHATC

SEQ ID NO: 5 - protein, 651 aa, Cimex lectularius
MLVVLSLALVALAHAGGPAHYRTASGAVGVLDGSLGQTHEETQKGYAGDSVSQFTSNVQGAHSYSTNSFSRTTNGGVAVGAPAVAAVRTVGPAVSYATPAVAALRGASFNHLGYSSGLGLSSGLGYGSGVRYGAGLGYGSGLRYGSGLGYGSGLRYGSGLGYGSGLSYGAGLGYGSGLRYGAGLGYGSGVSYGAGLGYTAVSPAVTTVHAAPAVTTVHAAPAVASLRSSTYHGSGYTGAGYASAAPLVSGLRTSSVSYSSPAVTTVHAAPAVAAVRTVAPAVSYAAPAVSYAAPAVASVRSSTYHGNGYTGASYAAPAVSAVRTVAPAVSYAAPAVSYAAPAVSYAAPAVASVRSSTYHGNGYTGASYAAPAVAAVRTSSVSYSSPAVTTVHAAPAVAAVRTVTPAVSYAAPAVASVRSSSYHGNGYTGVGYGAVAPAVSYAAPAVAAVRNVTPAVSYSAPAVTTVHAAPAVAAVRTVAPAVSYAAPAVASVRSSSYHGNGYTGVGYGAVAPAVSYAAPAVAAVRTVSTPAVSYAAPAVTTVHATPAFSYGAPAFRTVSAGPAVNTYSSSSYLGNAYGGLGYAAHVSPVAVGYSAPSVRTYSTATPLFKSYAAAPALTTYAAHSAPLAVGFSAAPSVSHATFSSLGSSYSF

SEQ ID NO: 6 - protein, 14 aa, Cimex lectularius
SGLRTSSVSYSSPA

SEQ ID NO: 7 - protein, 14 aa, Cimex lectularius
HSYSTNSFSRTTNG

SEQ ID NO: 8 - protein, 280 aa, Cimex lectularius
MLVVLSLALVALAHAGGPAHYRTASGAVGVLDGSLGQTHEETQKGYAGDSVSQFTSNVQGAHSYSTNSFSRTTNGGVAVGAPAVAAVRTVGPAVSYATPAVAALRGASFNHLGYSSGLGLSSGLGYGSGVRYGAGLGYGSGLRYGSGLGYGSGLRYGSGLGYGSGLSYGAGLGYGSGLRYGAGLGYGSGVSYGAGLGYTAVSPAVTTVHAAPAVTTVHAAPAVASLRSSTYHGSGYTGAGYASAAPLVSGLRTSSVSYSSPAVTTVHAAPAVAAVRTVAP

(19) United States
(12) Patent Application Publication
(10) Pub. No.: US 2018/0244734 A1
(43) Pub. Date: Aug. 30, 2018

(54) NOVEL PROTEINS AND DETECTION METHODS

(71) Applicant: AIRMID HEALTH GROUP LIMITED, Dublin 2 (IE)

(72) Inventors: Natasha GORDON, Dublin (IE); Luke O'SHAUGHNESSY, Athleague, Roscommon (IE); Bruce MITCHELL, Dublin 2 (IE)

(21) Appl. No.: 15/755,371

(22) PCT Filed: Aug. 31, 2016
(86) PCT No.: PCT/EP2016/070532

§ 371 Date: Feb. 26, 2018

(30) Foreign Application Priority Data
Aug. 31, 2015 (EP) 15183177.3

(51) Int. Cl. C07K 14/435; G01N 33/53

(52) U.S. Cl. CPC C07K 14/43563; C07K 16/18; C07K 2317/33; G01N 33/5308

(57) ABSTRACT

An isolated protein from Cimex lectularius comprising an amino acid sequence selected from any one of SEQ ID No. 1 or 5 or fragment thereof or an isolated polypeptide from Cimex lectularius comprising an amino acid sequence selected from any one or more of SEQ ID No. 2, SEQ ID No. 3, SEQ ID No. 6 and SEQ ID No. 7 or fragment thereof for use in generating antibodies to detect the presence of antigens specific for Cimex lectularius wherein the antibodies are capable of detecting Cimex lectularius antigen in all stages of Cimex lectularius development from egg, nymphs, moults to mature male and female adults at antigen concentrations of less than 2 μg/ml.

### INTRODUCTION

The present invention relates to novel Cimex lectularius proteins and methods to detect the presence of bed bugs in an environment such as a home or commercial setting.

Bed bugs are reddish-brown, wingless, obligate hemophagous requiring a blood meal for eclosion. They are flat, oval and elongated in shape, approximately 5-7 mm in length and have 3 pairs of legs. Bed bugs are insects that survive on the blood of humans and other warm-blooded hosts. Bed bugs are attracted to heat, scented bedbug excrement and carbon dioxide given off by warm-blooded mammals, birds, and themselves. Sleeping areas, mattresses, box springs, nightstands, headboards, are an ideal environment for bedbugs to hide. Bed bugs most likely hide during daylight hours in dark enclosed sites and have a preference for cracks and crevices in fabric, wood, and gritty sandpaper like surfaces. They are often found hiding in crevices and cracks of beds and furniture.

Bed bugs feed on blood, are active at night and bite any areas of exposed skin. A number of adverse health effects may occur due to bed bug bites, including skin rashes, allergic reactions and/or mental distress.

In recent years there has been a dramatic increase in the incidence of bed bug infestations in Europe, Australia and North America. This is due partly to the increase of global travel and migration. Other explanations include an increase in pesticide resistance/changes to the pest control management and use of second-hand furniture. The rise in the bed bug population has contributed to an increase in bed bug bites and related conditions.

The spread of bed bugs leading to infestation occurs through two forms of dispersal: 1—Active dispersal which requires the bed bugs to migrate from room to room through ventilation ducts, holes in walls and also pipes by their own means such as walking. Active dispersal can be a result of pesticide dispersal, intersexual conflict or host stimuli and 2—Passive dispersal or human transportation, where bed bugs are carried on clothing or within luggage and furniture to a new location, birds and bats also accommodate passive dispersal of bed bugs.

Bed bugs progress through 5 instar phases before reaching adulthood. At each stage during this process a blood meal is needed to induce moulting for progression to the next stage to be made. Female bed bugs can lay 200-500 eggs throughout their lifetime.

There are two main species of bed bugs which interact exclusively with humans

1. Cimex lectularius (the common bed bug)

2. Cimex hemipterus (which is found in tropical areas)

The two species C. lectularius and C. hemipterus appear to thrive in a mixed population and there has even been an increase in the prevalence of C. hemipterus being found in Australia and the UK indicating that whilst each species still has a defined niche the lines are becoming blurred.

Currently there is limited information on bed bugs at the molecular level as until very recently only transcriptomics had been performed. Transcriptomics is the study of transcribed RNA present in various cell or tissue types at a particular time. These expressed RNAs are then made into a cDNA library for molecular analysis. These studies have resulted in a large number of complete and partially complete mRNA sequence reads from three different bed bug strains (Richmond—Pesticide resistant, Harlan—Pesticide susceptible, and a pesticide exposed strain from an Ohio apartment.

There is a real need to provide a means to rapidly and easily detect the presence of bed bugs.

### Statements of Invention

According to the invention there is provided an isolated protein from Cimex lectularius comprising an amino acid sequence selected from any one of SEQ ID No. 1 or 5 or fragment thereof or an isolated polypeptide from Cimex lectularius comprising an amino acid sequence selected from any one or more of SEQ ID No. 2, SEQ ID No. 3, SEQ ID No. 6 and SEQ ID No. 7 or fragment thereof for use in generating antibodies to detect the presence of antigens specific for Cimex lectularius wherein the antibodies are capable of detecting Cimex lectularius antigen in all stages of Cimex lectularius development from egg, nymphs, moults to mature male and female adults at antigen concentrations of less than 20 μg/ml.

One embodiment of the invention provides a recombinant truncated polypeptide from Cimex lectularius protein comprising an amino acid sequence selected from any one of SEQ ID No. 4 or 8 or fragment thereof for use in generating antibodies to detect the presence of antigens specific to Cimex lectularius wherein the antibodies are specific for Cimex lectularius antigen in all stages of Cimex lectularius development from egg to mature adult and at antigen concentrations of less than 20 μg/ml.

In one embodiment of the invention the antibodies generated are monoclonal antibodies.

In one embodiment of the invention the antibodies are capable of detecting Cimex lectularius antigen at concentrations of less than 10 μg/ml.

In another embodiment of the invention the antibodies generated are able to detect antigens specific to both Cimex lectularius and Cimex hemipterus.

In one embodiment of the invention the protein isolated from Cimex lectularius is an insoluble protein.

In one embodiment of the invention the isolated protein or polypeptide from Cimex lectularius is for use in allergen testing.

According to the invention there is provided a method for the detection of Cimex lectularius or Cimex hemipterus in a sample comprising the steps of;
contacting the sample with a monoclonal antibody to form a monoclonal antibody-polypeptide complex; and
contacting the monoclonal antibody-polypeptide complex with a further monoclonal antibody labelled with a reporter agent which binds to the complex thereby detecting the presence of Cimex lectularius or Cimex hemipterus in the sample.

Preferably the sample comprises Cimex lectularius or Cimex hemipterus at any stage of male or female bed bug development from eggs, nymphs, moults or adults. Most preferably the monoclonal antibodies are specific for Cimex lectularius antigen in all stages of Cimex lectularius development from egg, nymphs, moults to mature male and female adults.

According to the invention there is also provided a method for the detection of Cimex lectularius or Cimex hemipterus in a sample comprising;
a lateral flow membrane;
a first area positioned at a first, lower, end of the lateral flow membrane for receiving a test sample, wherein the first area comprises a monoclonal antibody specific for Cimex lectularius or Cimex hemipterus, the antibody being labelled with a reporter agent;
a second area positioned at a second, upper, end of the lateral flow membrane comprising an immobilised control polypeptide; and
a third area positioned between the first and second area, wherein the third area comprises an immobilised monoclonal antibody specific for Cimex lectularius or Cimex hemipterus.

Preferably the monoclonal antibody is present at a concentration of less than 10 mg/ml. Most preferably the monoclonal antibody is present at a concentration of less than 5 mg/ml.

In one embodiment of the invention the detectable labelling agent is selected from any one or more of a latex antibody conjugate or a gold antibody conjugate.

In another embodiment of the invention the monoclonal antibody is labelled with any one or more of HRP or biotin.

According to the invention there is provided a diagnostic kit for detecting the presence of Cimex lectularius or Cimex hemipterus in a sample comprising:—
at least one solid surface on which a monoclonal antibody specific for an amino acid sequence selected from any one or more of SEQ ID No. 1, 2, 3, 4, 5, 6, 7 or 8 or fragment thereof is immobilized;
contacting the sample with the monoclonal antibody under conditions that allow binding of said antibody to Cimex lectularius or Cimex hemipterus antigens in the sample; and
comparing the amount of antibody that binds to the sample to a control value and therefrom determining the presence or absence of Cimex lectularius or Cimex hemipterus in the sample.

According to the invention there is provided a rapid detection method for determining the presence or absence of Cimex lectularius or Cimex hemipterus comprising a diagnostic kit of the invention.

One embodiment of the invention provides an isolated protein from Cimex lectularius comprising an amino acid sequence selected from any one of SEQ ID No. 1 or 5 or fragment thereof or isolated polypeptide from Cimex lectularius comprising an amino acid sequence selected from any one or more of SEQ ID No. 2, SEQ ID No. 3, SEQ ID No. 6, and SEQ ID No. 7 or fragment thereof.

One embodiment of the invention provides a recombinant truncated polypeptide from Cimex lectularius protein comprising an amino acid sequence selected from any one of SEQ ID No. 4 or 8 or fragment thereof.

### BRIEF DESCRIPTION OF THE INVENTION

The invention will be more clearly understood from the following description thereof with reference to the accompanying drawings in which:—

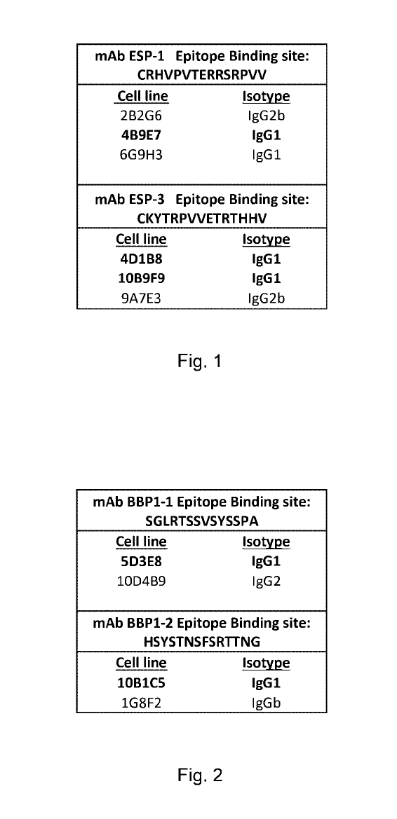

FIG. 1 is a table showing six mouse IgG monoclonal antibodies produced against bed bug egg shell protein epitopes 1 (ESP) (SEQ ID No. 2) and 3 (SEQ ID No. 3) from mouse hybridoma cell lines, (generated by GenScript USA Inc);

FIG. 2 is a table showing four mouse IgG monoclonal antibodies produced against bed bug protein 1 epitopes 1 (BBP1)(SEQ ID No. 6) and 2 (SEQ ID No. 7) from mouse hybridoma cell lines, (generated by GenScript USA Inc);

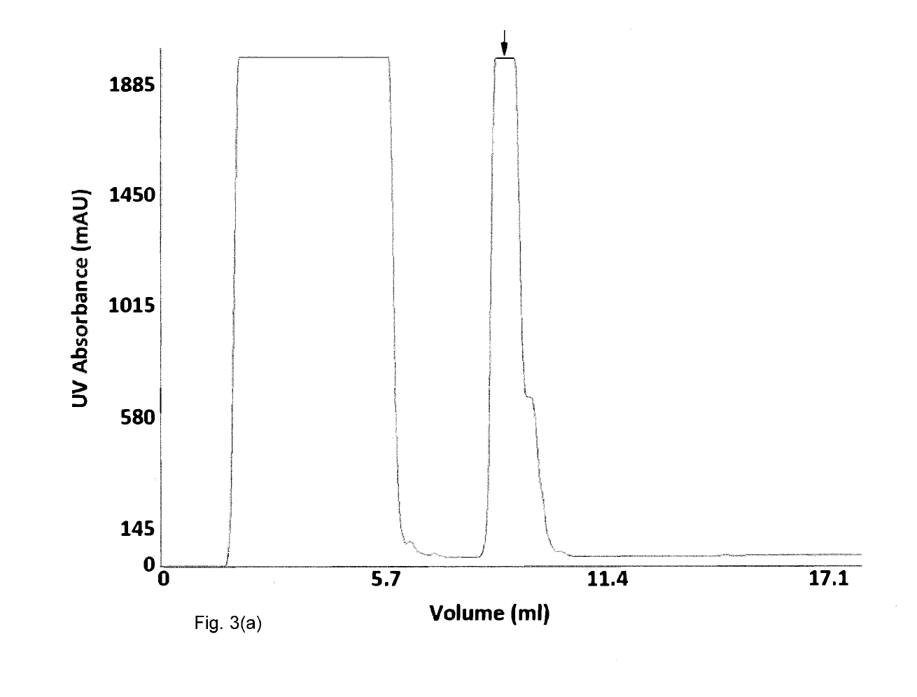

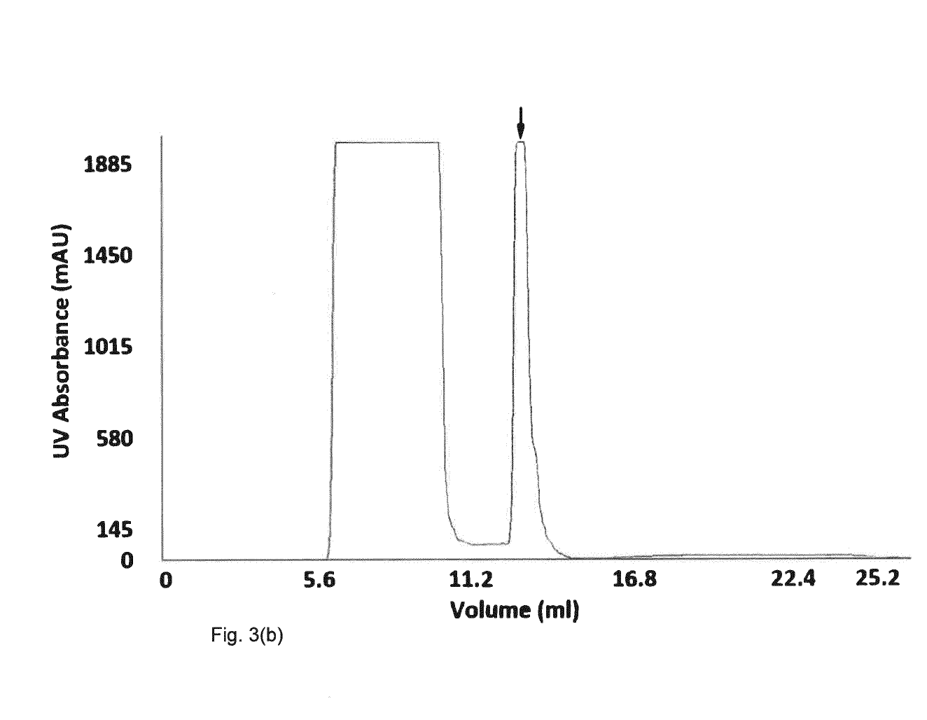

FIG. 3 is an affinity purification profile of the ESP monoclonal antibodies purified by a Hi-Trap protein G column (GE Healthcare), (a) shows the monoclonal antibody mAb ESP-1 [4139E7] and (b) the mAb ESP-3 [4D1B8 and 10B9F9]. The monoclonal antibodies were eluted with 0.1M glycine, pH 6.5, using a flow rate of 1 ml/minute.

The arrow denotes the start of the elution. Peak fractions were pooled and concentrated using centricon device;

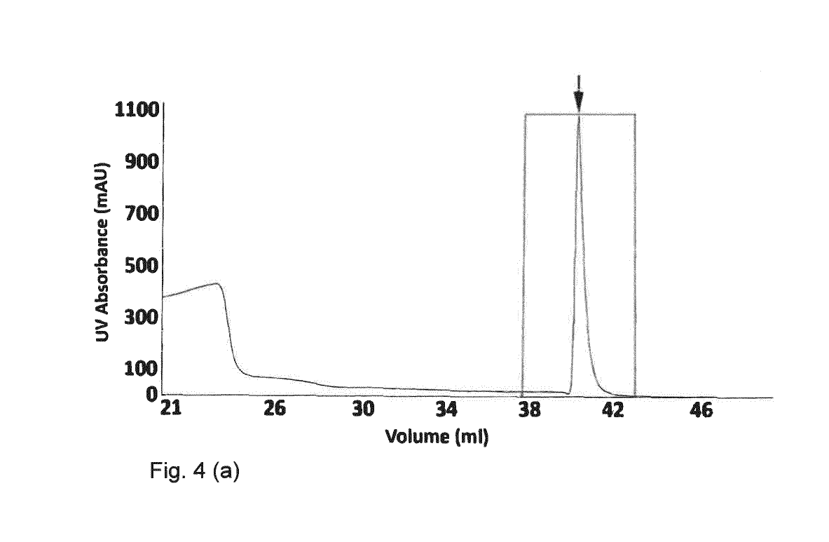

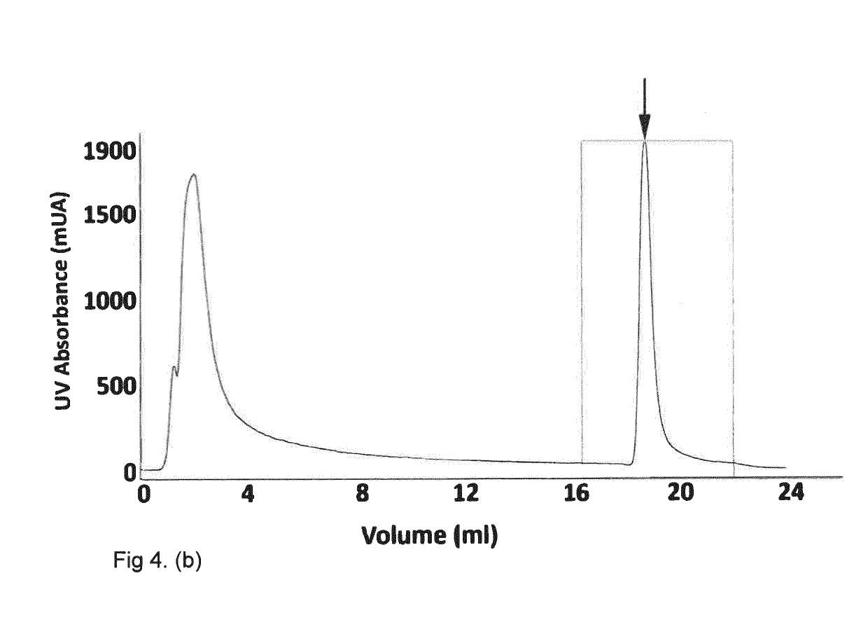

FIG. 4 is an affinity purification profile of the BBP1 monoclonal antibodies purified by a Hi-Trap protein G column (GE Healthcare), (a) shows the monoclonal antibody mAb BBP1-1 [5D3E8] and (b) the mAb BBP1-2 [10B105]. The monoclonal antibodies were eluted with 0.1M glycine, pH 6.5, using a flow rate of 1 ml/minute. The arrow denotes the start of the elution. Peak fractions were pooled and concentrated using centricon device;

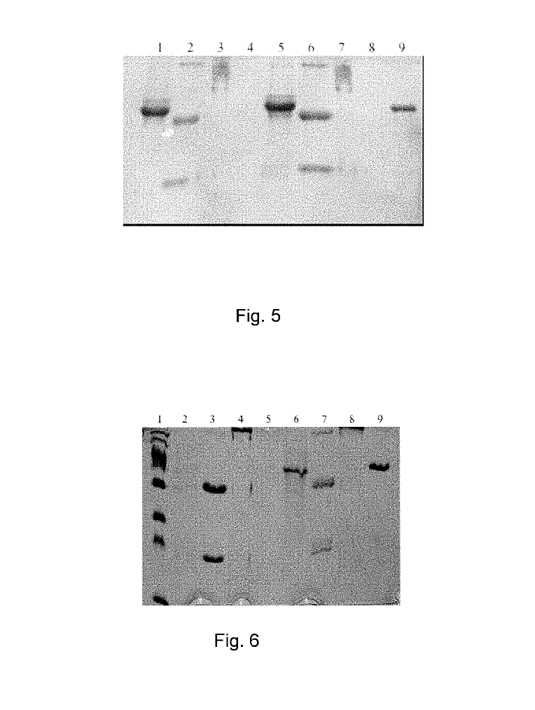

FIG. 5 is an SDS-PAGE analysis of the protein G column affinity purified mAb ESP-1 and mAb ESP-3 monoclonal antibodies in FIG. 3.

Lane 1: Flow through mAb ESP-1, Lane 2: Reduced mAb ESP-1, Lane 3: Non-reduced mAb ESP-1, Lane 4: blank, Lane 5: Flow through mAb ESP-3, Lane 6: Reduced mAb ESP-3, Lane 7: Non-reduced mAb ESP-3, Lane 8: blank, Lane 9: BSA control (66.5 kDa);

FIG. 6 is an SDS-PAGE analysis of the protein G column affinity purified mAb BBP1-1 and mAb BBP1-2 monoclonal antibodies in FIG. 4

Lane 1: Prestained Protein Marker, Lane 2: Hybridoma medium mAb BBP1-1, Lane 3: Reduced mAb BBP1-1, Lane 4: Non-reduced mAb BBP1-1, Lane 5: Blank, Lane 6: Hybridoma medium mAb BBP1-2, Lane 7: Reduced mAb BBP1-2, Lane 8: Non-reduced mAb BBP1-2, Lane 9: BSA Control (66.5 kDa);

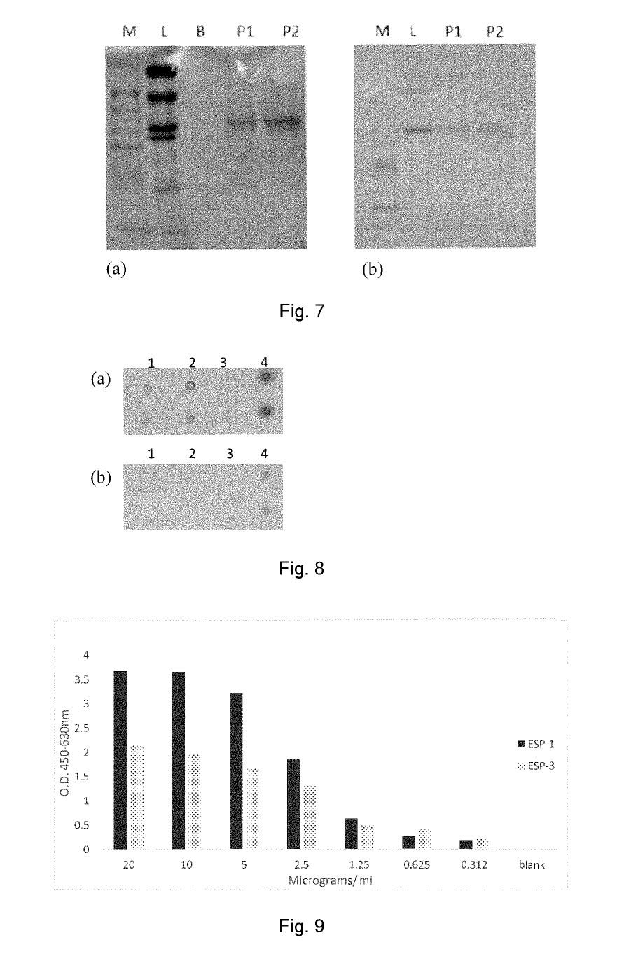

FIG. 7 (a) shows purified native ESP protein run on an SDS-PAGE protein gel.

FIG. 7 (b) is a western blot probed using the rabbit polyclonal antibodies anti-ESP-3 to identify the ESP protein.

FIG. 8 are dot blots showing (a) the biotin labelling of mAb ESP-1 [4B9E7] versus (b) unlabelled mAb ESP-1 [4B9E7]. Spot 1: Neat bed bug egg lysate; spot 2: ⅕ dilution bed bug egg lysate; Spot 3: BSA control; and Spot 4: Neat antibody dotted onto the membrane (control);

FIG. 9 is a graph of the results of an ELISA assay showing the optimum coating concentration for the monoclonals mAb ESP-1 and mAb-ESP-3. The secondary conjugate Goat anti-mouse HRP was diluted 1/5000. The optimum coating concentration was found to be ˜5-10 μg/ml for mAb ESP-3;

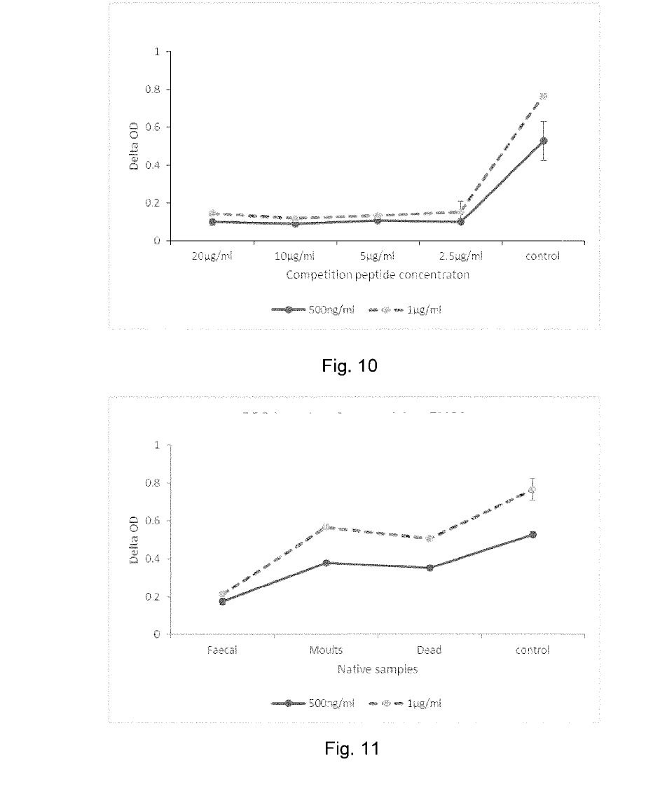

FIG. 10 is a graph showing the results of a competitive BBP1 polyclonal ELISA detecting BBP1 peptide. Delta OD was collected by measuring the absorbance at 450 nm and 630 nm;

FIG. 11 is a graph showing the results of a competitive BBP1 polyclonal ELISA detecting BBP1 protein in faecal, moults and dead bed bug samples. Delta OD was collected by measuring the absorbance at 450 nm and 630 nm;

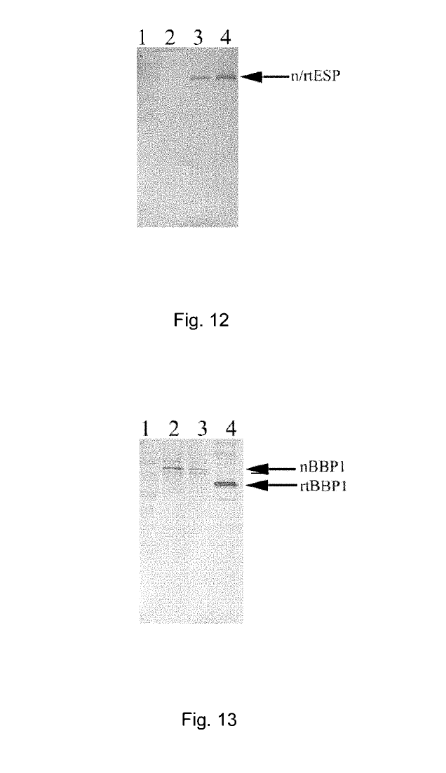

FIG. 12 is a western blot showing the detection of enriched native and recombinant truncated ESP (rtESP)

Lane 1: Molecular weight standard ladder, Lane 2: Bed bug egg lysate, Lane 3: immune-precipitated ESP protein, Lane 4: rtESP. The arrow denotes native ESP protein at 36.3 kDa and rtESP at 40 kDa;

FIG. 13 is a western blot showing the detection of enriched native and recombinant truncated BBP1 (rtBBP1)

Lane 1: Molecular weight standard ladder, Lane 2: Bed bug lysate, Lane 3: immune-precipitated BBP1 protein, Lane 4: rtBBP1. The arrow denotes native BBP1 protein at 63 kDa and rtBBP1 at 39 kDa;

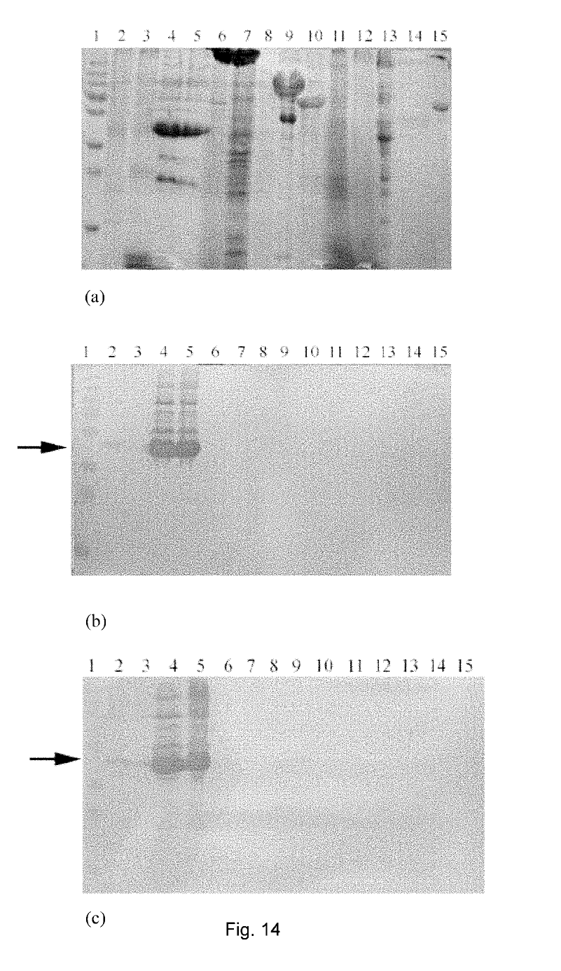

FIG. 14 is (a) an SDS-PAGE gel (b) a western blot probed with mAb ESP-1 and (c) a western blot probed with mAb ESP-3 to show insect lysate cross reactivity and antibody specificity to the ESP protein.

Lane 1: Prestained Protein Marker, Lane 2: Harlan strain bed bug whole insect lysate, Lane 3: London Lab strain bed bug whole insect lysate, Lane 4: Harlan strain bed bug whole egg lysate, Lane 5: London Lab strain bed bug whole egg lysate, Lane 6: House spider whole insect lysate, Lane 7: House moth whole insect lysate, Lane 8: Cockroach whole insect lysate, Lane 9: Cockroach egg whole lysates, Lane 10: Woodlouse Whole insect lysate, Lane 11: Centipede whole insect lysate, Lane 12: House dust mite whole lysate, Lane 13: House fly whole insect lysate, Lane 14: Bee whole insect lysate and Lane 15: BSA control (66.5 kDa); The arrow denotes native ESP protein at 36.3 kDa;

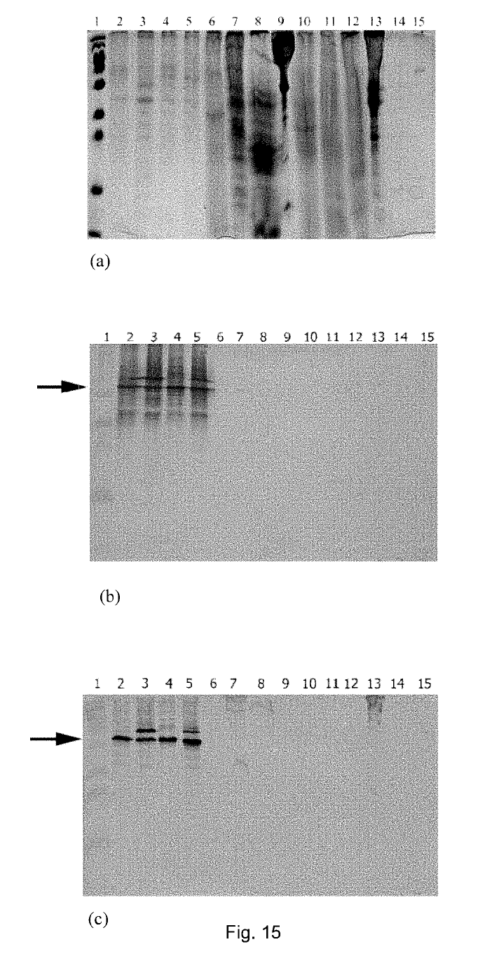

FIG. 15 is (a) an SDS-PAGE gel (b) a western blot probed with mAb BBP1-1 and (c) a western blot probed with mAb BBP1-2 to show insect lysate cross reactivity and antibody specificity to the BBP1 protein.

Lane 1: Prestained Protein marker, Lane 2: Harlan strain bed bug whole insect lysate, Lane 3: London Lab strain bed bug whole insect lysate, Lane 4: Harlan strain bed bug moults lysate, Lane 5: London Lab strain bed bug moults lysate, Lane 6: House spider whole insect lysate, Lane 7: House moth whole insect lysate, Lane 8: Cockroach whole insect lysate, Lane 9: Cockroach egg whole lysates, Lane 10: Woodlouse Whole insect lysate, Lane 11: Centipede whole insect lysate; Lane 12: House dust mite whole lysate, Lane 13: House fly whole insect lysate, Lane 14: Bee whole insect lysate and Lane 15: BSA control (66.5 kDa). The arrow denotes native BBP1 protein at 63 kDa.

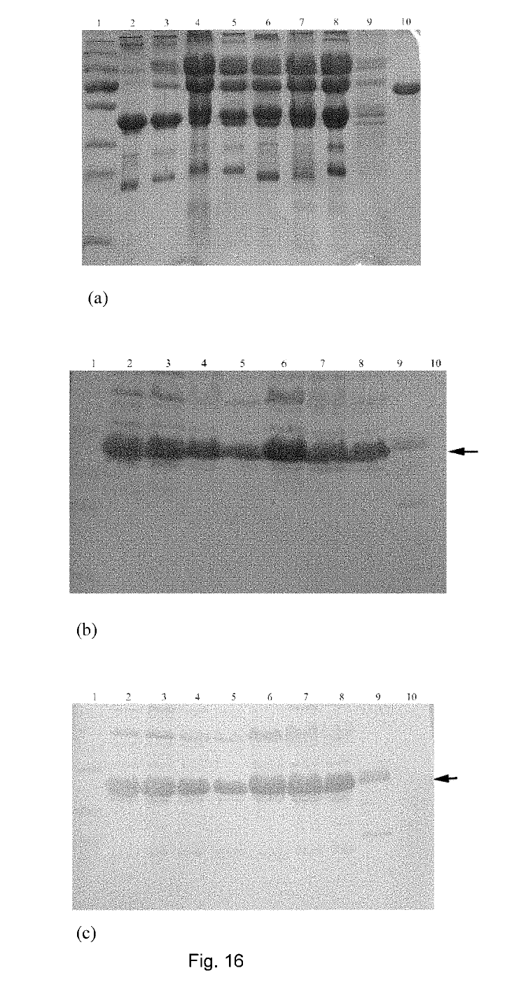

FIG. 16 (a) an SDS-PAGE gel (b) a western blot probed with mAb ESP-1 and (c) a western blot probed with mAb ESP-3 to show the detection of Cimex lectularius from different geographical regions and Cimex hemipterus

Lane 1: Prestained Protein Marker, Lane 2: Harlan strain (American—Cimex lectularius), Lane 3: London lab strain (Insecticide susceptible—Cimex lectularius), Lane 4: London Field strain (Pyrethroid resistant—Cimex lectularius), Lane 5: Kenyan Field Strain (Cimex lectularius), Lane 6: Kenya tropical Field Strain (Cimex hemipterus), Lane 7: German Lab Strain (Insecticide susceptible—Cimex lectularius), Lane 8: Sweden Field Strain (Cimex lectularius), Lane 9: Purified Native ESP, Lane 10: BSA (66.5 kDa). The arrow denotes native ESP protein at 36.3 kDa.

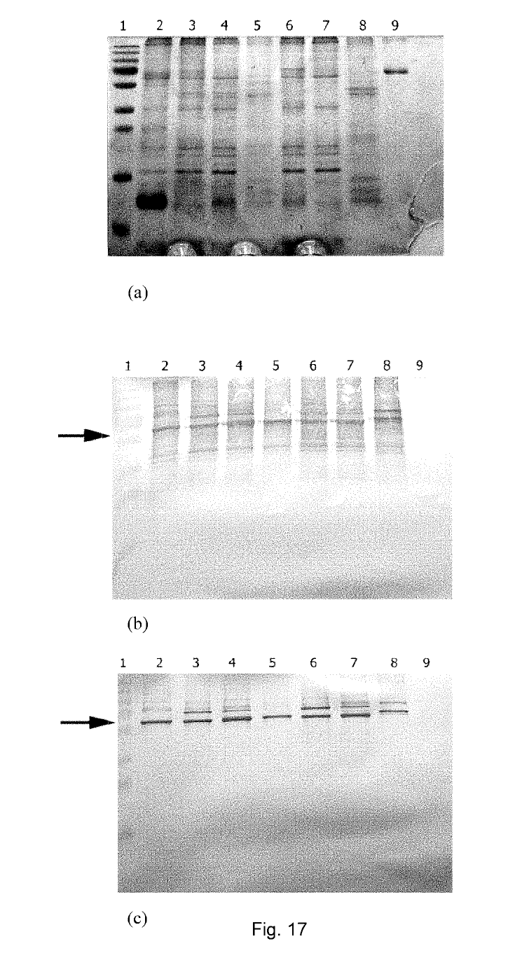

FIG. 17 is (a) an SDS-PAGE gel (b) a western blot probed with mAb BBP1-1 and (c) a western blot probed with mAb BBP1-2 to show the detection of Cimex lectularius from different geographical regions and Cimex hemipterus

Lane 1: Prestained Protein Marker, Lane 2: Harlan strain (American—Cimex lectularius), Lane 3: London lab strain (Insecticide susceptible—Cimex lectularius), Lane 4: London Field strain (Pyrethroid resistant—Cimex lectularius), Lane 5: Sweden Field Strain (Cimex lectularius), Lane 6: Kenyan Field Strain (Cimex lectularius), Lane 7: German Lab Strain (Insecticide susceptible—Cimex lectularius), Lane 8: Kenya tropical Field Strain (Cimex hemipterus), Lane 9: BSA (66.5 kDa). The arrow denotes native BBP1 protein at 63 kDa.

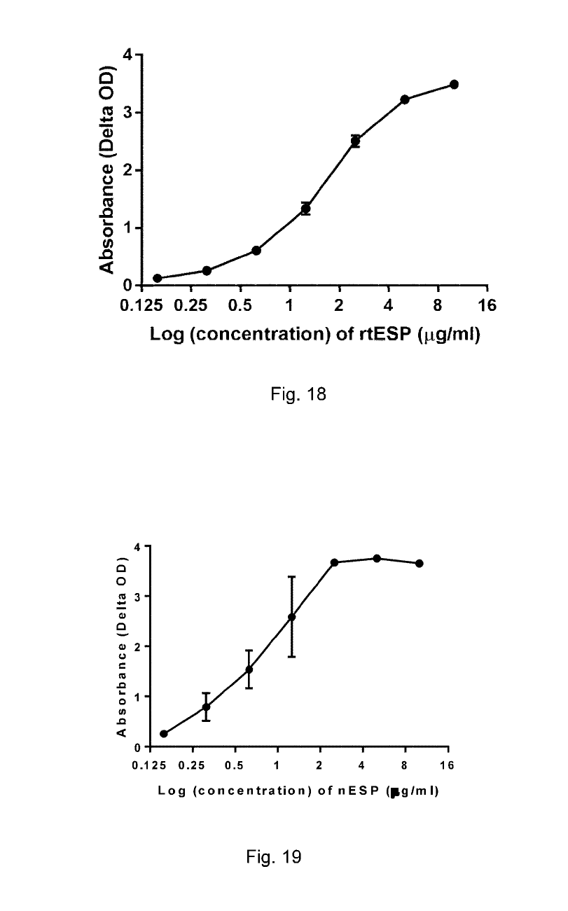

FIG. 18 is a rtESP standard curve of the results of a capture ELISA assay showing the binding profile for mAb ESP-1 [4139E7] and mAb ESP-1-HRP antibody, Delta OD was collected by measuring the absorbance at 450 nm and 630 nm;

FIG. 19 is a graph of the results of a capture ELISA assay showing the binding profile for nESP ELISA titration and it's correlation to rtESP, Delta OD was collected by measuring the absorbance at 450 nm and 630 nm;

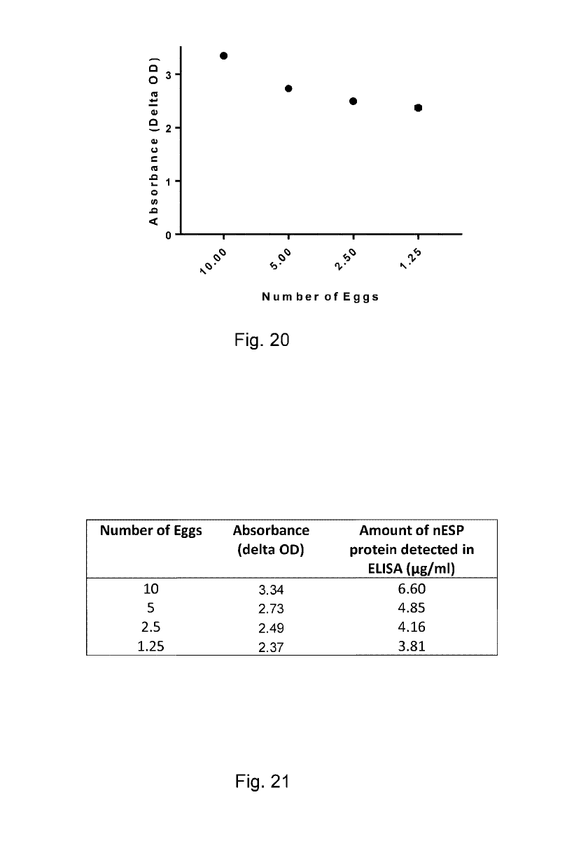

FIG. 20 is a graph of the limit of detection of a capture ELISA for nESP, Delta OD was collected by measuring the absorbance at 450 nm and 630 nm;

FIG. 21 is a table showing the limit of detection of the capture ELISA for n ESP;

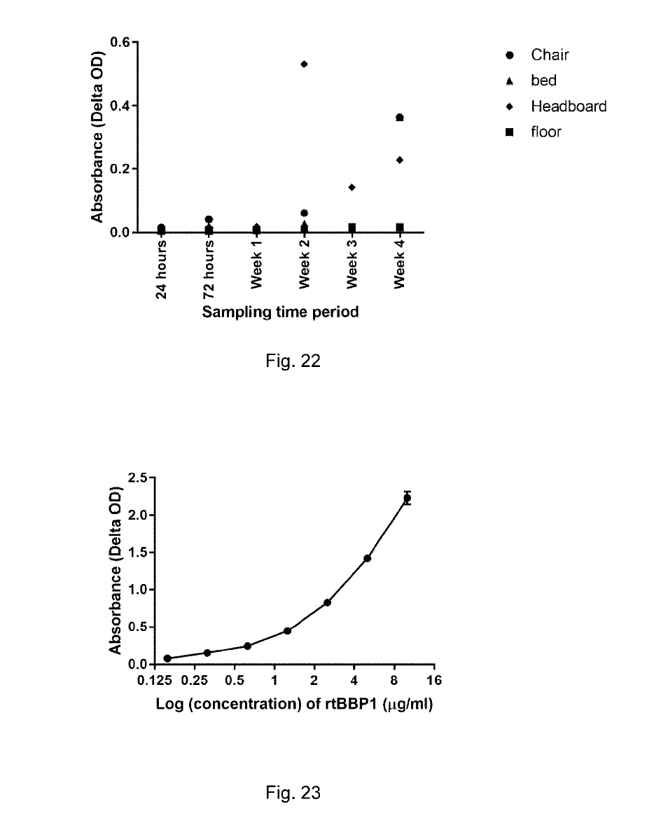

FIG. 22 is a graph showing the detection of nESP in samples taken at different time periods (24 hours, 72 hours, week 1, 2, 3 and 4) from an environmental test chamber;

FIG. 23 is a rtBBP1 standard curve of the results of a capture ELISA assay showing the binding profile for mAb BBP1-1 and mAb BBP1-1-HRP antibody, Delta OD was collected by measuring the absorbance at 450 nm and 630 nm;

FIG. 24 is a graph of the results of a capture ELISA assay showing the binding profile for nBBP1 ELISA titration and it's correlation to rtBBP1, Delta OD was collected by measuring the absorbance at 450 nm and 630 nm;

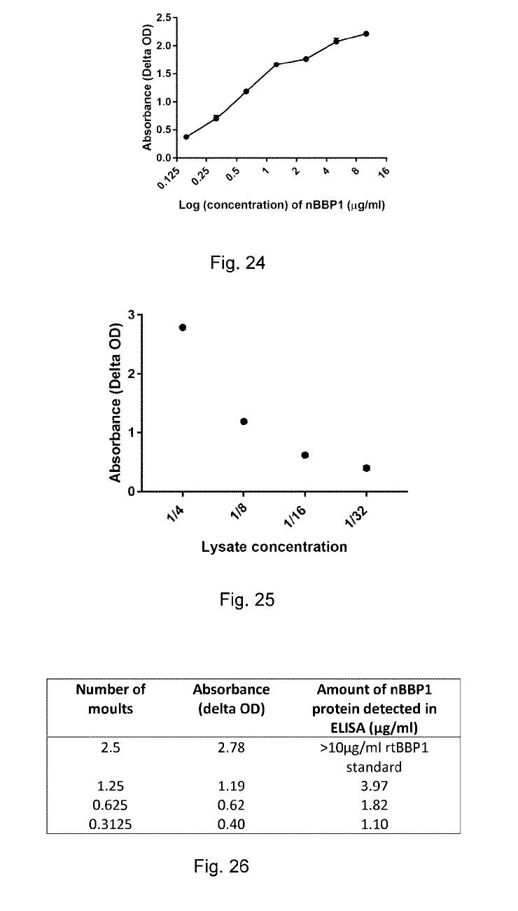

FIG. 25 is a graph of the limit of detection of a capture ELISA for nBBP1 in terms of number of moults, Delta OD was collected by measuring the absorbance at 450 nm and 630 nm;

FIG. 26 is a table showing the limit of detection of the capture ELISA for n BBP1;

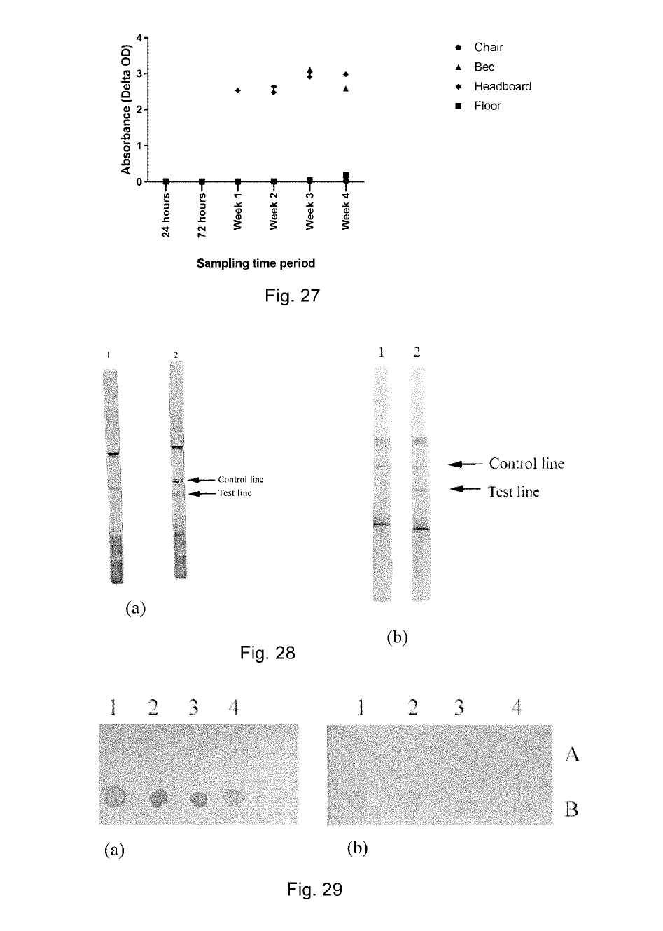

FIG. 27 is a graph showing the detection of nBBP1 in samples taken at different time periods (24 hours, 72 hours, week 1, 2, 3 and 4) from an environmental test chamber, Delta OD was collected by measuring the absorbance at 450 nm and 630 nm;

FIG. 28 shows a lateral flow test against Bed bug egg lysate (a) and BBP1 (b) Lane 1: Negative control (8M Urea lysis buffer), Lane 2: Extracted bed bug egg lysate (a); and Positive bed bug chamber sample (b)

FIG. 29 is a dot blot showing (a) mAb BBP1-1 (b) anti human IgE detection of rtBBP1, Row A: Lane 1: 0.5 mg/ml BSA, Row B: Lane 1: 4 μg rtBBP1, Lane 2: 2 μg rtBBP1, Lane 3: 1 μg rtBBP1, Lane 4: 0.5 μg rtBBP1;

### DETAILED DESCRIPTION

The rapid infestation of bed bugs around the world has led to growing concerns for the human health implications.

Bed bugs feed on human blood. The bites inflicted by bed bugs can create a dermatological reaction, which can cause irritation for a considerable time after the bite occurs. The first indication of a bite usually occurs with the person not knowing exactly where or when the insect bit them. This creates the situation whereby a person may not know where they were bitten. It may have been at a hotel, over a friend's house, the couch or their bed. In many instances, there is no reaction for many months or until sightings of the bed bugs are seen or the excrement and blood stains are found on the surface of where they are feeding frequently. The fear of bed bugs can lead to anxiety and stress.

The pandemic worldwide increase in bed bugs highlights the need for an improved means of detecting the presence of bed bugs. If the presence of bed bugs is confirmed action can then be taken to eliminate the insects. Alternatively if the absence of bed bugs is confirmed a person can sleep easier.

Others have described limited bed bug detection systems. Tolley M P et al (“Identification of bed bug (Cimex Lectularius) surface deposited residues as a means of development of bed bug detection devices” URL:http://esa.confex.com/esa/2011/webprogram/paper54779.html) describe a system using a bed-bug specific salivary antigen, nitrophorin. The system depends on antigens specific to human blood voided in bed bug excreta. Sensitivity of the results appears to be a function of the bed bug infestation ie. Low populations yield no detectable results.

WO2013/130613 (SRI International) describe a polyclonal antibody detection system for ectoparasites. The system relies on polyclonal antibodies generated from an immunogen comprising a whole animal extract. The samples prepared were found to be remarkably soluble even without the addition of any detergents. The specificity of polyclonal antibodies generated to whole ectoparasitic immunogen would not be very high and would only detect soluble proteins.

In complete contrast the present invention provides novel Cimex lectularius proteins which were identified and isolated. The isolated proteins are insoluble proteins. The proteins are easily extracted from bed bug eggs, moults, and exoskeletons and whole insects, however a detergent is required to solubilise the isolated protein. An exoskeleton may be from live or dead insects at any life stage. Antibodies raised to the isolated proteins and polypeptides were found to be able to detect the presence of antigen from all stages in the life cycle of a male or female bed bug from egg to 1^{st}-5^{th} instar forms to mature adults. In addition the presence of antigen was detected from moults which bed bugs can shed up to five times throughout their lifespan. The antibodies were found to be very specific with no cross reactivity with other closely related ectoparasites. Antibody epitope regions on the proteins were identified and found to be very specific for the proteins. Antibodies raised to the epitope regions were found to detect antigen in samples at very low antigen concentrations.

Surprisingly the antibodies raised were capable of detecting proteins in both the common bed bug species Cimex lectularius and Cimex hemipterus which is found in tropical areas. The assay systems of the present invention using the specific antibodies of the present invention have global application in being able to identify bed bugs from different geographical regions. Due to climate changes the occurrence of Cimex hemipterus is more common in more temperate climates. The present invention provides sensitive assay systems which may be used all over the world to detect the presence of bedbugs.

One novel protein was found to be present within the eggshell of Cimex lectularius eggs, egg shell protein (ESP).

A second novel protein was found to be present within all life stages of the Cimex lectularius insect, bed bug protein 1 (BBP1). BBP1 was detected in faecal deposits, moults, bed bug eggs, adult bed bugs, 1^{st}-5^{th} instar forms [u1] of Cimex lectularius.

The novel isolated egg shell protein (ESP) was found to be a 363 amino acid protein comprising SEQ ID No. 1. The identified antibody epitope regions comprise SEQ ID Nos. 2 and 3. The egg shell protein (ESP) was produced as a truncated protein as a way of stabilising ESP protein from degradation seen in the native ESP protein. Predictive structural software (Phyre2, scratch protein predictor) indicated a two domain structure for the ESP protein. The truncated version of ESP was developed to incorporate the predicted C terminal domain of the protein starting at V114-C363. The rtESP protein made up of 251 amino comprising SEQ ID No. 4 was prepared to be used as a standard in ESP assays.

The novel isolated protein found in all life stages of Cimex lectularius (bed bug protein 1—BBP1) was found to be a 651 amino acid protein comprising SEQ ID No. 5. The identified antibody epitope regions comprise SEQ ID No. 6 and 7. BBP1 is a large protein with a highly repetitive amino acid sequence, for this reason when constructing a recombinant protein, a truncated version of BBP1 was produced. 280 amino acids of the protein N terminal region were used to produce the truncated recombinant protein comprising SEQ ID No. 8.

The novel isolated Cimex lectularius proteins were found to have no PFAM domains similar to proteins of other insects. The eggshell protein comprising SEQ ID No. 1 was found to be present only in the eggshell of Cimex lectularius. The bed bug protein 1—BBP1 comprising SEQ ID No. 5 was found to be present in faecal deposits and in moults, eggs, adults and 1^{st}-5^{th} instar forms of Cimex lectularius.

According to the invention variants or fragments of the protein or polypeptide sequences include conservative substitutions or modifications such that they retain the immunogenic properties of the proteins. Variants may also or alternatively contain other modifications including the deletion or addition of amino acids that have minimal influence on the antigenic properties of the polypeptide.

Native eggshell (ESP protein) and bed bug protein 1 (BBP1 protein) protein was purified by differential protein extraction and checked by western blot methods for antibody specificity.

Antibody epitope regions were identified on the proteins and sensitive detection assays have been developed using both monoclonal and polyclonal antibodies specific for the novel Cimex lectularius proteins. The isolation and identification of the novel proteins and raising antibodies to the novel proteins provides potential application for the detection of bed bugs in samples. The assays may be used to detect the presence of Cimex lectularius in all life stages.

The sensitivity of antibodies raised to the proteins was found to be very high. The antibodies were able to detect antigen concentration levels in samples less than 20 μg/ml. The antibodies were able to detect antigen concentration levels as low as 1-3 μg/ml.

Assay systems were developed using the antibodies raised to the antibody epitope regions on the novel proteins.

Detection assays using the antibodies of the present invention have valuable applications in many areas. Detection assays using the antibodies allows one to quickly and easily determine the presence or absence of bed bugs in a sample. Domestic users or hotels will be able to confirm with certainty the absence of bed bugs in the environment. Likewise individual travelers will be able to carry an easy to use assay kit to confirm whether a hotel room or bed is free of bed bugs. Having a detection assay kit with rapid easy read results would be very beneficial in quickly determining the presence or absence of bed bugs in an environment. Different types of detection assays may be developed such as for example ELISA assays or lateral flow tests or any other rapid assay platform. The antibodies of the present invention may be employed within assay formats, surface presented be it on array, nanoparticle or membrane and/or presented in a suspension format.

A lateral flow immunochromatographic detection method in the form of a dipstick test was used to detect the presence of bed bugs and was found to provide a rapid and highly sensitive detection method. (FIG. 28) In use samples are taken from the area being tested and may be placed in a diluent. The sample pad with the monoclonal attached is dipped into the sample diluent and the visualisation of a line indicates the presence of bed bugs.

Other formats for using the antibodies disclosed herein to detect the presence of bed bugs in a sample will be apparent to those skilled in the art.

The occurrence of allergies to bed bugs is on the rise and can lead to significant discomfort and ill health. Sensitivity towards bed bug bites appears to increase with repeated exposure, however bites and their subsequent reactions are often misdiagnosed. The ability to be able to determine whether a person had a specific allergy to bed bugs would be very valuable.

It was found that the novel proteins of the present invention may be used in allergen testing. Bed bug proteins elicit an IgE immune response following a bite or exposure. This response can result in many different symptoms including welts and asthma. Skin prick testing (SPT) demonstrates an allergic response to a specific allergen. SPT using the novel proteins of the present invention may be used to confirm the presence of an allergy to bed bugs. Specific diagnosis will help in treatment options and also help to prevent reoccurrences in symptoms through avoidance.

The invention will be further illustrated by the following Examples.

### Example 1—Extraction of Native ESP from Cimex lectularius Eggs

Bed bug eggs were collected 3 days post feeding and allowed to hatch. Hatched eggs were resuspended in urea lysis buffer (8M urea, 2M thiourea, 25 mM Tris, pH8.0, 150 mM NaCl, 1 mM EDTA, 1 mM EGTA and 1% Triton X-100) and sonicated in 1 minute bursts 3 times. Samples were centrifuged at 27 000×g for 10 minutes to separate the soluble and insoluble fractions of the sample.

The yield of native protein obtained from 250 eggs was approximately 6.2 mg of total purified native protein.

### Example 2—Extraction of Native BBP1 from Cimex lectularius Whole Bodies and Moults

Dead bed bug insects and moults were resuspended in urea lysis buffer (8M urea, 2M thiourea, 25 mM Tris, pH8.0, 150 mM NaCl, 1 mM EDTA, 1 mM EGTA and 1% Triton X-100) and vortexed in 1 minute bursts for 5 minutes. Samples were centrifuged at 27 000×g for 10 minutes to separate the soluble and insoluble fractions of the sample.

The yield of native protein obtained from 10 moults was approximately 30 μg of total purified native protein.

The novel proteins extracted and isolated were found to be insoluble proteins as they are not successfully extracted using PBS alone. The use of a detergent is required for both proteins to be fully solublised.

### Example 3—Generation of Mouse Monoclonal Antibodies (mAb) and Selection of Clones

Six mouse IgG monoclonal antibodies (mAb) against bed bug egg shell protein epitopes (ESP) 1—SEQ ID No. 2 and 3—SEQ ID No. 3 and four mouse IgG mAbs against bed bug protein 1 epitopes 1 (BBP1) SEQ-ID No. 6 and 2—SEQ ID No. 7 were produced from mouse hybridoma cell lines, (generated by GenScript USA Inc, on a commercial basis). Combining two clones for mAbESP-3 significantly increased the sensitivity of the immune reaction. The tables for the selected monoclonal antibodies are shown in FIGS. 1 and 2.

The hybridoma cells were expanded in DMEM+L glutamine+ antibiotic supplement with 10% FBS which was reduced to 2% FBS once the hybridoma cells were stable in T175 tissue culture flasks. The hybridoma cells were grown for antibody production until the medium turned yellow and the cells were dead (˜2 weeks). The supernatant was collected by centrifugation at maximum speed (4 300×g) for 30 mins at 4° C.

mAb were purified from hybridoma cell supernatants by ammonium sulphate precipitation (50%) overnight at 4° C. The precipitated antibodies were re-suspended in PBS and dialysed/buffer exchanged to remove salt. The mAb ESP-1 [4B9E7], mAb ESP-3 [4D1B8 & 10B9F9], mAb BBP1-1 [5D3E8] and BBP1-2 [10B105] were further purified using a Hi-Trap protein G column (GE Healthcare) according to the manufacturer's instructions. Briefly, the dialysed ammonium sulphate precipitated mAb were filtered through 0.45 μm filters. These were then passed over a 1 ml Protein G column (GE Healthcare) Unbound proteins were washed off the column with PBS (5 column volumes×three times). The mAb were eluted by glycine-HCl (0.1 M, pH=6.5), concentrated and then dialyzed against PBS (pH=7.5) at 4° C. overnight. The absorbance during the purification process was monitored at a wavelength of 280 nm, using the AKTA Start FLPC system. The concentrations of purified mAb was calculated at OD280 nm.

FIG. 3 shows the elution of the purified monoclonal antibody to ESP on a Protein G column (a) mAb ESP-1 and (b) mAb ESP-3. FIG. 4 shows the elution of the purified monoclonal antibody to BBP1 on a Protein G column (a) mAb BBP1-1 and (b) mAb BBP1-2.

The purity of the mAb was assessed by SDS-PAGE under reducing and non-reducing conditions. FIG. 5 shows the mAb ESP-1 and mAb ESP-3 purification SDS-PAGE. FIG. 6 shows the mAb BBP1-1 and mAb BBP1-2 purification SDS-PAGE.

### Example 4—Generation of Polyclonal Antibodies

Polyclonal antibodies to the ESP protein were generated by GenScript USA Inc, on a commercial basis. In brief two peptides were generated for ESP, ESP-1 comprising SEQ ID No. 2 and ESP-3 comprising SEQ ID No. 3 and one peptide for BBP1, BBP1-2 comprising SEQ ID No. 7. The peptides were conjugated to a carrier protein and used to immunize 2 rabbits per peptide. The polyclonal antibodies were affinity purified and checked via ELISA.

FIG. 7 (a) shows purified native ESP protein run on an SDS-PAGE protein gel. FIG. 7 (b) is a western blot probed using the rabbit polyclonal antibodies anti-ESP-3 to identify the ESP protein.

### Example 5—Biotinylation of ESP-1 [4B9E7] mAb

The monoclonal antibody to ESP was biotinylated to amplify the antibody to ESP protein signal.

Purified mAb ESP-1 [4B9E7] was dialysed against several changes of carbonate buffer [0.1 M sodium carbonate buffer (NaHCO₃/Na₂CO₃) pH 9.5 containing 0.1% NaN₃] at 2-8° C. After dialysis, the protein concentration was adjusted to 2 mg/ml. NHS-D-Biotin (Sigma) was dissolved in DMSO immediately prior to use (protecting solution from light) at a concentration of 2 mg/ml. Using a volume equal to 10% of the total volume of the immunoglobulin solution, the NHS-D-Biotin solution was added in portions to the monoclonal antibody solution with gentle stirring, and this was incubated at room temperature for 4 hours on a rotatory wheel. The reaction solution was dialysed against several changes of PBS buffer (0.01 M sodium phosphate, 0.15 M sodium chloride, pH 7.4, containing 0.1% NaN₃) at 4° C. to remove any unbound biotin. After dialysis, the biotinylated mAb ESP-1 [4B9E7] was stored in the dark at −20° C. (as biotin is light sensitive).

The biotinylation efficiency was checked via dot blot analysis (FIG. 8). It was found that biotinylation improved the efficiency of the monoclonal antibody.

### Example 6—Capture ELISA to Determine Optimum Coating Concentration

A 96 well ELISA plate was coated with a serial dilution (20-0.312 μg/ml) of mAb ESP-1 or mAb ESP-3 at 100 μl/well in 50 mM Carbonate buffer pH 9.6 and incubated overnight at room temperature. The coated ELISA plate was washed 3 times with PBST (PBS+0.05% Tween-20) and blocked (10% sucrose, 1% BSA in carbonate buffer pH 9.6) for 2 hours at room temperature. After washing, goat anti-mouse antibody at 1 in 4000 dilution was added at 100 μl/well and incubated for one hour at room temperature. The ELISA plate was washed 4 times with PBST and visualized using 100 μl/well of ultra TMB ELISA detector reagent. H₂SO₄ was used to stop the reaction after 5 minutes. The absorbance's at 450 nm and 630 nm were measured and used to calculate the delta OD value. (FIG. 9)

### Example 7—Assay to Detect Cimex lectularius Whole Insects and Moults in a Sample Using a BBP1 Polyclonal Competition ELISA

BBP1 Biotinylation

BBP1 peptide (SEQ ID No. 7) was biotinylated with EZ-link sulfo-NHS biotin (Thermo fisher) as per manufacturer's instructions. In brief BBP1 peptide was mixed with a 20 fold excess of sulfo-NHS biotin and incubated for 30 minutes at room temperature with rotation. Sample was then passed over a C18 column (Pierce) to remove free biotin from reacted BBP1-biotin.

Sample extraction for BBP1 ELISA

5 moults, 5 dead bed bugs and 1 cm of faecal paper was incubated in 500 μl TBS Saline (25 mM Tris, 150 mM NaCl, pH 7.2 0.1% BSA, 0.05% Tween 20) for 2.5 hours with rotation. Sample was used neat.

Competitive ELISA

Neutravidin plates (Pierce) were used as per manufactures instructions. The ELISA plate was coated with 100 μl BBP1— biotin peptide (2.5 μg/ml) in Tris buffered Saline (25 mM Tris, 150 mM NaCl, pH 7.20.1% BSA, 0.05% Tween 20) and incubated for 2 hours at room temperature. The ELISA plate was washed 3 times with TBS prior to the addition of 50 μl peptide (various concentrations) or sample (neat) and 50 μl of anti-BBP1 polyclonal antibody (500 ng/ml or 1 μg/ml final). The plates were incubated for 1 hour at room temperature and washed 3 times before the addition of 100 μl anti-rabbit-HRP at a dilution of 1 in 2500. TMB ELISA detector reagent was used to visualise the signal and H₂SO₄ was used to stop the reaction after 5 minutes. Delta OD was collected by measuring absorbance at 450 nm and 630 nm.

The assay was able to detect low levels of BBP1 protein. The BBP1 antibody was able to detect the synthesised BBP1 peptide at levels as low as 2.5 μg/ml (FIG. 10). The BBP1 antibody was also able to detect native BBP1 protein in neat samples extracted from faecal paper, moults and dead bed bugs (FIG. 11).

### Example 8—Recombinant Protein Purification

Genes for ESP and BBP1 (rtESP and rtBBP1) truncated proteins were generated as g blocks (integrated DNA technologies) and cloned into a pET32a vector. Proteins were expressed in either origami(DE3)pLysS (novagen) for rtESP—pET32a or BL21(DE3)pLysS (novagen) for rtBBP1—pET32a cells according to manufacturer's instructions. Recombinant protein was expressed in inclusion bodies. Bacteria pellets were resuspended in lysis buffer (5% triton X-100, 0.1 mM PMSF, phosphate buffer (25 0 mM phosphate, pH 8.0, 300 mM NaCl)) and sonicated at 50% maximum power (Syclon, ultrasonic homogenizer) until lysate was clear. Inclusion bodies were pelleted at 27 000×g for 15 minutes then washed once in wash buffer I (50 mM Tris, pH8.0, 0.5% Triton X-100, 1 mM DTT phosphate buffer) and then wash buffer II (50 mM Tris, pH8.0, 1 mM DTT, phosphate buffer). Protein pellets were then resuspended in protein resuspension buffer (6M GuHCl, 10 mM Imidazole, phosphate buffer).

rtBBP1 was further purified using a mAb BBP1-1 CNBr sepharose column. The rtBBP1 suspension was passed over the column and washed with 10 column volumes of PBS. Protein was eluted from the column in 100 mM glycine pH 3 and buffered with 1M tris pH 8.0.

### Example 9—Enrichment and Detection of Native ESP and BBP1

Native ESP and BBP1 proteins were immuno-precipitated from bed bug egg lysate and bed bug whole lysate using mAb ESP-3/mAb BBP1-1 CNBr sepharose column in TSA (10 mM Tris, pH 8.0, 140 mM NaCl). Unbound proteins were removed by washing the beads six times with wash buffer (500 mM NaCl, 10 mM Tris pH8.0, 1% triton X-100, 1% Sodium deoxycholate, 0.1% SDS) and TSA. The enriched samples were separated on an 12.5% SDS-PAGE under reducing conditions. Protein samples were transferred onto a nictrocellulose membrane.

The nitrocellulose membrane was blocked with 5% milk powder in PBST. The membrane was probed with mAb ESP-1 or mAb BBP1-1 at a 1 in 1000 dilution for 1 hour. The nitrocellulose membrane was washed three times with PBST, this was followed by probing with anti-mouse—HRP at a 1 in 2000 dilution for 1 hr. The membrane was washed three times with PBST and the signal was visualised with DAB (3,3′-diaminobenzidine) Substrate.

The results of detection of the native and recombinant truncated ESP are shown in FIG. 12. The arrow denotes native ESP protein at 36.3 kDa and rtESP at 40 kDa.

The results of detection of the native and recombinant truncated BBP1 are shown in FIG. 13. The arrows denote native BBP1 protein at 63 kDa and rtBBP1 at 39 kDa.

### Example 10—Immunoblot to Determine Cross Reactivity and Antibody Specificity of the Monoclonal Antibodies to ESP and BBP1 Protein

The cross reactivity of the monoclonal antibodies to ESP and BBP1 was checked against a range of insects and their eggs which are associated with the domestic setting. The insects were collected and then frozen in liquid nitrogen and crushed with a pestle and mortar.

The crushed lysates were resuspended in urea lysis buffer and protease inhibitors, pH8. The proteins were extracted by gentle mixing for 2 hrs. Following this the lysates were centrifuged at 27 000×g to remove the soluble supernatant. The soluble supernatants were removed and filtered through a spin filter column. The protein concentration was determined via Bradford assay and dilute protein lysates were concentrated using a centricon device with a low molecular weight cut-off. Samples were then loaded onto a 12.5% SDS-PAGE gel and run under reducing conditions. The results are shown in FIG. 14 (a).

Following electrophoresis the proteins were transferred onto a nitrocellulose membrane for probing with each of the purified mAb.

The nitrocellulose membrane was blocked with 5% milk powder in PBST. The membrane was probed with mAb ESP-1, mAb ESP-3 at a 1 in 1000 dilution and mAb BBP1-1 and mAb BBP1-2 at a 1 in 2000 dilution for 1 hour. The nitrocellulose membrane was washed three times with PBST, this was followed by anti-mouse—HRP at a 1 in 2000 dilution for 1 hr. The membrane was washed three times with PBST and the signal was visualised with DAB (3,3′-diaminobenzidine) Substrate.

FIG. 14 (b) shows the results of probing with mAb ESP-1. FIG. 14 (c) shows the results of probing with mAb ESP-3. The arrow denotes native ESP protein at 36.3 kDa.

The monoclonal antibodies were found to be very specific for both the Harlan strain bed bug whole egg lysate and London Lab strain bed bug whole egg lysate (FIGS. 14(b) and (c) lanes 4 and 5). No cross reactivity was found with the other eggs tested.

### Example 11—Immunoblot to Determine Cross Reactivity and Antibody Specificity of the Monoclonal Antibodies to BBP1 Protein

As in the previous example the cross reactivity of the monoclonal antibodies to BBP1 were checked against a range of insects which are associated with the domestic setting.

FIG. 15 (a) shows the samples run under reducing conditions on a 12.5% SDS-PAGE gel. FIG. 15 (b) shows the results of probing with mAb BBP1-1. FIG. 15 (c) shows the results of probing with mAb BBP1-2. The arrow denotes native BBP1 protein at 63 kDa.

The monoclonal antibodies were found to be specific for both the Harlan strain bed bug whole insect lysate and moults lysate and London Lab strain bed bug whole lysate and moults lysate (FIGS. 15(b)(c) lanes 2 to 5). No cross reactivity was found with any other lysates from other insects that were tested.

### Example 12—Immunoblot to Investigate Detection of Cimex lectularius from Different Regions and Cimex hemipterus

A range of Cimex lectularius bed bugs and bed bug eggs collected from different regions and from a tropical Kenyan bed bug species (Cimex hemipterus) were extracted as previously described in urea lysis buffer. The samples were centrifuged at 27 000×g for 10 minutes to separate the soluble and insoluble sample fractions. The concentration of the soluble fraction was determined by Bradford assay and all samples were diluted to the same concentration and separated on a 12.5% SDS-PAGE under reducing conditions. Proteins were transferred onto a nitrocellulose membrane and western blot was run as previously described.

The SDS-PAGE results under reducing conditions for ESP are shown in FIG. 16(a). A western blot showing the detection using mAb ESP-1 is shown in FIG. 16(b) and using mAb ESP-3 is shown in FIG. 16 (c). The arrow denotes native ESP protein at 36.3 kDa.

The SDS-PAGE results under reducing conditions for BBP1 are shown in FIG. 17(a). A western blot showing the detection using mAb BBP1-1 is shown in FIG. 17 (b) and using mAb BBP1-2 is shown in FIG. 17 (c). The arrow denotes native BBP1 protein at 63 kDa.

Surprisingly it was found that the monoclonal antibodies were specific for ESP and BBP1 in not only Cimex lectularius but also Cimex hemipterus (FIG. 16, Lane 6 and FIG. 17, Lane 8)

### Example 13—Field Trials

An environmental test chamber was set up to replicate a hotel room, it contained a bed, a bedside locker and a chair. The temperature (23° C.) and relative humidity (45%±5%) of the chamber were controlled throughout the study. Fed bed bugs (mixed life stages) were introduced into the chamber at weekly intervals. The number of bed bugs introduced was doubled at each successive week to monitor the exponential effect of an active infestation.

An artificial feeder was introduced into the chamber after 1 week to allow the bed bugs to feed once inside the chamber. Vacuum samples were taken from areas of known bed bug activity at 24 and 48 hours and at weeks 1, 2, 3 and 4 of the study.

### Example 14—Capture ELISA

A 96 well ELISA plate was coated with 100 μl/well of mAb ESP-3 or mAb BBP1-1 (5 μg/ml) in 50 mM Carbonate buffer pH 9.6 and incubated overnight at 4° C. The coated ELISA plate was washed 3 times with PBST (PBS+0.05% Tween-20) and blocked (10% sucrose, 1% BSA in carbonate buffer pH 9.6) for 2 hours at room temperature. Extracted bed bug samples were diluted ¼ into PBST, 1% BSA then serially diluted (1:2) onto ELISA plates and incubated for 1 h at room temperature on a plate shaker platform. After washing, mAb ESP-1-HRP or mAb BBP1-2-HRP antibody at 1 in 500 dilution was added at 100 μl/well and incubated for one hour at room temperature. The ELISA plate was washed 3 times with PBST and visualized using 100 μl/well of ultra TMB ELISA detector reagent. H₂SO₄ was used to stop the reaction after 10 minutes. The absorbance's at 450 nm and 630 nm were measured and used to calculate the delta OD value.

An rtESP protein standard curve is shown in FIG. 18. An nESP ELISA titration and correlation to rtESP is shown in FIG. 19. The results of an ELISA showing the limit of detection (LOD) is shown in FIG. 20. The antibodies were able to detect ESP protein present at concentrations less than 10 μg/ml. The assay was able to detect the ESP protein in a sample containing as few as 1 to 2 bed bug eggs. (FIG. 21)

Results from the detection of bed bug eggs within the controlled chamber at different time periods using the ELISA are shown in FIG. 22.

An rtBBP1 protein standard curve is shown in FIG. 23. An nBPP1 ELISA titration and correlation to rtBBP1 is shown in FIG. 24. The results of an ELISA showing the limit of detection (LOD) by way of insect moults is shown in FIG. 25. The antibodies were able to detect BBP1 protein present at concentrations less than 10 μg/ml. The assay was able to detect the BBP1 protein in a sample containing as few as 1 moult from a bed bug. (FIG. 26).

Results from the detection of bed bugs within a controlled chamber at different time periods using the ELISA are shown in FIG. 27.

### Example 15—Generation of Latex—mAb Conjugates

100 μl of 400 nm, carboxylated Microspheres in a 10% w/v solution were washed three times with activation buffer (50 mM MES pH6.0). The microsphere carboxyl groups were activated for 30 minutes at room temperature with 4.8 mM 1-ethyl-3-[3-dimethylamniopropyl] carbodiimide (EDC) and 48 mM N-hydroxysulfosuccinimide (sulfo-NHS). The microspheres were then washed twice with activation buffer before the addition of 50 mg/g of antibody (mAb ESP-1 or mAb BBP1-2) in activation buffer. The microspheres:antibody solution was mixed overnight at 4° C. The 1% microsphere solution was quenched with 30 μl of ethanolamine for 30 minutes at room temperature then blocked with blocking buffer (50 mM Tris, pH8.0 and 1% casein) for 3 hours at room temperature with rotation. Following two rounds of washing using blocking buffer, the microspheres were resuspended in blocking buffer at a final concentration of 1%. Microspheres were sonicated at intervals through out the process to ensure monodispersal of particles.

Different concentrations of antibody may be applied to the microspheres and different antibodies to the ESP and BBP1 proteins may be used. The method is not limited to the addition of BSA binding step to increase antibody concentration levels.

### Example 16—Lateral Flow Test Preparation

The lateral flow test consists of a dip stick model that may also be used in cassettes or other rapid detection formats. A HiFlow plus membrane (Millipore) was attached to a membrane backing card and sprayed with a test line of either mAb ESP-3 (5 mg/ml, 2.5 mg/ml, 1 mg/ml)) or mAb BBP1-1 (6 mg/ml, 3 mg/ml, 1 mg/ml) and a control line of anti-mouse (250 ug/ml).

Latex conjugates (mAb ESP-1 and mAb BBP1-2) prepared as described above were diluted to a concentration of 0.05%-0.1% in conjugation buffer (2% fish skin gelatin, 1% sucrose, 1% Triton X-100, 1% Tween 20, 1.5 mg/ml PEG 4000) and were then applied to a glass fibre membrane and dried for 72 hours at 37° C. Glass fibre membranes were also used as samples pads and were pre-treated with the conjugation buffer and dried under the same conditions as the conjugate pad.

The lateral flow test was constructed by slightly overlapping the absorbance pad onto the upper section of the sprayed membrane and the conjugate pad onto the lower section of the sprayed membrane. The sample pad slightly overlaps the conjugate pad in the lower section of the test. The membranes were dried at 37° C. for 24 hours before being cut into 75 mm×4 mm test strips. Lateral flow strips were tested on bed bug chamber samples and egg lysates. Urea lysis buffer was used as a control. The positive results are shown in FIG. 28 (a)—ESP and FIG. 28 (b)—BBP1.

Other sampling methods may be used for example a swab or wipe of the area may be taken and the swab or wipe added to diluent into which the lateral flow strips are subsequently dipped.

### Example 17—Generation of Gold—mAb Conjugates

Gold—mAb Conjugates were Prepared for Use in Lateral Flow Tests.

Unbuffered colloidal gold was buffered to pH 7.5 before 10 μg/ml of mAb (mAb ESP-3) was added to solution and rotated for 2 hours. Gold mAb conjugate was blocked for 2 hours at room temperature with BSA blocking solution. Gold mAb conjugates were washed extensively

Gold mAb conjugates (mAb ESP-3) were used at an OD range of 1.5-2.5 in conjugation buffer (2% fish skin gelatin, 1% sucrose, 1% Triton X-100, 1% Tween 20, 1.5 mg/ml PEG 4000, 1.5 mg/ml PVPK-30) and were then applied to a glass fibre membrane and dried for 72 hours at 37° C. Glass fibre membranes were also used as samples pads and were pre-treated with the conjugation buffer and dried under the same conditions as the conjugate pad.

### Example 18—Allergen Testing

rtBBP1 was spotted onto a nitrocellulose membrane at titrating concentrations (4 μg/ml, 2 μg/ml, 1 μg/ml, 0.5 μg/ml), BSA (0.5 mg/ml) was used as a control. The nitrocellulose membrane was blocked with 5% milk powder in PBST. The membrane was probed with mAb BBP1-1 at a 1 in 1000 dilution or human sera for 2 hour. The nitrocellulose membrane was washed three times with PBST followed by anti-mouse—HRP at a 1 in 2000 dilution or anti-human IgE—peroxidase at a 1 in 500 dilution for 1 hr. The membrane was washed three times with PBST and the signal was visualised with DAB (3,3′-diaminobenzidine) Substrate. The results of the dot blot are shown in FIG. 29.

The present invention provides polyclonal and monoclonal antibodies capable of detecting both Cimex lectularius and Cimex hemipterus at very low concentration levels. The monoclonal antibodies have valuable application in point-of-use assays to detect antigens specific to bed bugs. Assay methods may include ELISA assays or lateral flow immunochromatographic tests. The user may vacuum, swab or wipe an area and insert the sample into a reader. Very low bed bug populations may be detected. Optional reagents may include a solution to solubilise bed bug samples from the collection device.

It will of course be understood that the invention is not limited to the specific details as herein described, which are given by way of example only, and that various alterations and modifications are possible without departing from the scope of the invention as defined in the appended claims.

1. An isolated protein from Cimex lectularius comprising an amino acid sequence selected from any one of SEQ ID No. 1 or 5 or fragment thereof or an isolated polypeptide from Cimex lectularius comprising an amino acid sequence selected from any one or more of SEQ ID No. 2, SEQ ID No. 3, SEQ ID No. 6 and SEQ ID No. 7 or fragment thereof for use in generating antibodies to detect the presence of antigens specific for Cimex lectularius wherein the antibodies are capable of detecting Cimex lectularius antigen in all stages of Cimex lectularius development from egg, nymphs, moults to mature male and female adults at antigen concentrations of less than 20 μg/ml.

2. A recombinant truncated polypeptide from Cimex lectularius protein as claimed in claim 1 comprising an amino acid sequence selected from any one of SEQ ID No. 4 or 8 or fragment thereof for use in generating antibodies to detect the presence of antigens specific to Cimex lectularius wherein the antibodies are specific for Cimex lectularius antigen in all stages of Cimex lectularius development from egg, nymphs, moults to mature male and female adults and at antigen concentrations of less than 20 μg/ml.

3. An isolated protein or polypeptide from Cimex lectularius as claimed in claim 1 wherein the antibodies generated are monoclonal antibodies.

4. An isolated protein or polypeptide as claimed in claim 1 wherein the antibodies are capable of detecting Cimex lectularius antigen at concentrations of less than 10 μg/ml.

5. An isolated protein or polypeptide from Cimex lectularius as claimed in claim 1 wherein the antibodies generated are able to detect antigens specific to both Cimex lectularius and Cimex hemipterus.

6. An isolated protein from Cimex lectularius as claimed in claim 1 wherein the protein is an insoluble protein.

7. An isolated protein or polypeptide from Cimex lectularius as claimed in claim 1 for use in allergen testing.

8. A method for the detection of Cimex lectularius or Cimex hemipterus in a sample comprising the steps of;
contacting the sample with a monoclonal antibody specific as claimed in claim 3 to form a monoclonal antibody-polypeptide complex; and
contacting the monoclonal antibody-polypeptide complex with a further monoclonal antibody labelled with a reporter agent which binds to the complex thereby detecting the presence of Cimex lectularius or Cimex hemipterus in the sample.

9. A method as claimed in claim 8 wherein the sample comprises Cimex lectularius or Cimex hemipterus at any stage of male or female bed bug development from eggs, nymphs, moults or adults.

10. A method as claimed in claim 8 wherein the monoclonal antibodies are specific for Cimex lectularius antigen in all stages of Cimex lectularius development from egg, nymphs, moults to mature male and female adults.

11. A method for the detection of Cimex lectularius or Cimex hemipterus in a sample comprising;
a lateral flow membrane;
a first area positioned at a first, lower, end of the lateral flow membrane for receiving a test sample, wherein the first area comprises a monoclonal antibody specific for Cimex lectularius or Cimex hemipterus as claimed in claim 3, the antibody being labelled with a reporter agent;
a second area positioned at a second, upper, end of the lateral flow membrane comprising an immobilised control polypeptide; and
a third area positioned between the first and second area, wherein the third area comprises an immobilised monoclonal antibody specific for Cimex lectularius or Cimex hemipterus.

12. A method as claimed in claim 10 wherein the monoclonal antibody is present at a concentration of less than 10 mg/ml.

13. A method as claimed in claim 10 wherein the monoclonal antibody is present at a concentration of less than 5 mg/ml.

14. A method as claimed in claim 10 wherein the detectable labelling agent is selected from any one or more of a latex antibody conjugate or a gold antibody conjugate.

15. A method as claimed in claim 10 wherein the monoclonal antibody is labelled with any one or more of HRP or biotin.

16. A diagnostic kit for detecting the presence of Cimex lectularius or Cimex hemipterus in a sample comprising:—
at least one solid surface on which a monoclonal antibody specific for an amino acid sequence selected from any one or more of SEQ ID No. 1, 2, 3, 4, 5, 6, 7 or 8 or fragment thereof is immobilized;
contacting the sample with the monoclonal antibody under conditions that allow binding of said antibody to Cimex lectularius or Cimex hemipterus antigens in the sample; and
comparing the amount of antibody that binds to the sample to a control value and therefrom determining the presence or absence of Cimex lectularius or Cimex hemipterus in the sample.

17. A rapid detection method for determining the presence or absence of Cimex lectularius or Cimex hemipterus comprising a diagnostic kit as claimed in claim 15.

18. An isolated protein from Cimex lectularius comprising an amino acid sequence selected from any one of SEQ ID No. 1 or 5 or fragment thereof or isolated polypeptide from Cimex lectularius comprising an amino acid sequence selected from any one or more of SEQ ID No. 2, SEQ ID No. 3, SEQ ID No. 6, and SEQ ID No. 7 or fragment thereof.

19. A recombinant truncated polypeptide from Cimex lectularius protein comprising an amino acid sequence selected from any one of SEQ ID No. 4 or 8 or fragment thereof.
